(19) United States
(12) Patent Application Publication
(10) Pub. No.: US 2008/0268551 A1
(43) Pub. Date: Oct. 30, 2008

(54) System and Method for Biological Assays

(76) Inventors: Christopher N. Bowman, Boulder, CO (US); Robert P. Sebra, Powhatan, VA (US); Kristi S. Anseth, Boulder, CO (US); Kristyn S. Masters, Madison, WI (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/721,968

(22) PCT Filed: Dec. 20, 2005
(86) PCT No.: PCT/US05/46822

§ 371 Date: Mar. 24, 2008

Related U.S. Application Data
(60) Provisional application No. 60/637,685, filed on Dec. 20, 2004.

(51) Int. Cl. G01N 33/549; G01N 33/544

(52) U.S. Cl. 436/532; 436/531

(57) ABSTRACT

The present invention provides polymers and microfluidic devices comprising a covalently attached substrate binding element, and methods for producing and using the same.

### CROSS-REFERENCE TO RELATED APPLICATIONS

This application claims the priority benefit of U.S. Provisional Application No. 60/637,685, filed Dec. 20, 2004, which is incorporated herein by reference in its entirety.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. F49620-02-1-0042 awarded by the Department of Defense.

### FIELD OF THE INVENTION

The present invention relates to polymers and microfluidic devices comprising a covalently attached substrate binding element, and methods for producing and using the same.

### BACKGROUND

Attaching (e.g., tethering) of biomolecules to surfaces is beneficial for numerous applications ranging from tissue engineering to biological assays. Proteins and oligopeptide moieties are now routinely immobilized on various material surfaces to control cellular interactions. Many biosensor technologies rely on methods that capture soluble molecules by surface-bound ligands (e.g., antibody-antigen interactions). Regardless of the biofunctionalization method used, retaining biological activity of the surface-bound molecule, while maintaining and enhancing selectivity toward the target analyte molecule, is important for various applications, e.g., diagnostic assays.

Most conventional antigen detection assays (e.g., standard enzyme-linked immunosorbant assays) rely on monolayer formation or physisorption methods to immobilize antibodies to material surfaces. However, this approach leads to drawbacks associated with antibody coating stability and uniformity and is often dependent on material properties, such as surface chemistry and roughness. Thus, recent research has focused on methods to covalently bind antibodies to material surfaces through conventional protein functional sites, such as amine and carboxy terminal groups. While these approaches reduce the possibility of antibody desorption, surface-bound antibodies and biomolecules, in general, often lose their activity and/or selectivity due to conformational restrictions, mobility restrictions, and/or mass transfer limitations. Furthermore, antibody activity is often reduced or lost due to protein unfolding or reduction of antigen binding sites due to the coupling process used.

Therefore, there is a need for a method for covalently attaching substrate binding elements.

### SUMMARY OF THE INVENTION

One aspect of the present invention provides a method for grafting (i.e., covalently attaching) a substrate binding element to a polymer using a radical reaction. The method generally involves using a polymer that comprises a first polymerizable functional group on its surface and coating the polymer surface with a substrate binding monomer to produce a polymerizable mixture. The substrate binding monomer comprises a substrate binding element that is covalently attached to a linker comprising a second polymerizable functional group. This polymerizable mixture is then polymerized to produce a polymer comprising covalently attached substrate binding monomer. In one embodiment, the method involves using a radical reaction to produce the polymer. In such embodiment, the functional groups present in the polymer surface and the substrate binding monomer are selected such that at least a portion of these functional groups react with one another via a radical reaction to generate the polymer comprising a covalently attached substrate binding element. In one particular embodiment, the polymer surface comprises an iniferter moiety, which can be readily cleaved to generated a reactive radical species.

Another aspect of the present invention provides a polymer produced from a monomer mixture comprising a thiol monomer and an olefinic monomer, said polymer comprising a surface bound substrate binding element. The substrate binding element is covalently attached to the polymer surface by a covalently attached linker.

Still another aspect of the present invention provides, a method for producing a polymer comprising a covalently attached substrate binding element. The method comprises polymerizing an admixture of a first monomer and a substrate binding monomer by a radical polymerization reaction to produce a polymer comprising a covalently attached substrate binding element. The first monomer comprises a first polymerizable functional group and the substrate binding monomer comprises a substrate binding element that is covalently attached to a linker comprising a second polymerizable functional group such that the radical polymerization reaction results in formation of a covalent bond between at least a portion of the first polymerizable functional group and at least a portion of the second polymerizable functional group by a radical polymerization process.

The polymer that is produced using some of the methods of the present invention comprise a covalently attached substrate binding element on the polymer surface. Other methods of the present invention provide polymers comprising a covalently attached substrate binding element on the polymeric bulk matrix as well as the polymer surface.

Yet another aspect of the present invention provides a microfluidic device comprising:
a polymer derived from a monomeric mixture comprising a thiol monomer and an olefinic monomer; and
a plurality of channels, wherein a surface of at least a portion of one of the channels comprises a surface bound substrate binding element, wherein said substrate binding element is covalently attached to said surface by a covalently attached linker.
   It should be appreciated that the microfluidic devices of the present invention can also include other features and geometries, such as wells, grooves, furrows, etc.

Another aspect of the present invention provides a device for use in a biological assay comprising a polymer produced from a monomer mixture comprising a thiol monomer and an olefinic monomer; and wherein at least a portion of the polymer surface comprises a covalently attached substrate binding element, wherein said substrate binding element is covalently attached to the polymer surface by a linker.

In some embodiments of the present invention, the linker comprises polyethylene glycol, poly(vinyl alcohol), poly(hydroxy methacrylate), poly(hydroxy acrylate), poly(urethane), poly(acrylamide), poly(amines), or a combination thereof. In one particular embodiment, the linker comprises polyethylene glycol.

In certain embodiments of the present invention, the substrate binding element is an antibody.

Still in other embodiments, the substrate binding element is an antigen.

In other embodiments, the substrate binding element is a cell.

Yet in some embodiments, the polymer comprises a plurality of surface bound substrate binding elements, where each of the substrate binding element has different binding affinity for different substrates.

Still in certain embodiments, each of the surface bound substrate element is covalently linked to a portion of the polymer.

In some embodiments, the polymer surface is photoreactive.

In certain embodiments, the polymer comprises a photoiniferter moiety.

Yet in other embodiments, the substrate binding element is photografted or photopolymerized to said polymer surface.

Still another aspect of the present invention provides a substrate binding monomer comprising a substrate binding element that is covalently attached to a linker comprising a polymerizable functional group.

### BRIEF DESCRIPTION OF THE DRAWINGS

FIG. 1 is a schematic illustration of covalently attaching a substrate binding element to a linker;

FIG. 2 is a schematic illustration of covalently attaching a substrate binding element to a polymer surface;

FIG. 3 is a schematic illustration showing an increase in antimer chain length with increasing exposure time;

FIG. 4 is a SDS-Page result showing an increase in the degree of acrylation and molecular weight with increasing amounts of ACRL-PEG-NHS in the reaction stoichiometry;

FIG. 5 is a graph of chromagenic intensity as a function of antigen concentration for a standard ELISA assay that uses adsorbed, unmodified antibodies (□) and adsorbed acrylated antibodies (O) for the detection of RAM antigen in PBS;

FIG. 6 shows chromagenic intensity of photografted square patterns of antibody-PEG acrylate monomer at various antigen concentration as compared control surfaces containing only PEG grafts (C1) and PEG grafts synthesized in the presence of non-acrylated antibody (C2);

FIG. 7 is a graph showing a surface bound antimer density with increasing exposure time;

FIG. 8 is a graph of chromagenic intensity as a function of antigen concentration using a standard ELISA assay (□) and a method of the present invention (O);

FIG. 9A is a schematic illustration of one embodiment of the present invention where an antibody that is covalently attached to a polymer through a linker is used in a sandwich immunoassay for rapid detection of a specific antigen;

FIG. 9B is a schematic illustration of a conventional sandwich assay where an antibody is attached directly to the polymer surface without any linker;

FIG. 10A is a plot of chromogenic intensity as a function of GLGN antigen concentration in PBS (⋄), 20% plasma (∘), and 20% whole blood (□) analyte;

FIG. 10B is a plot of chromogenic intensity of GLGN in a 20% plasma environment comparing results from the method of the present invention (□) and a conventional ELISA assay (∘);

FIG. 11A is a schematic illustration of a 3-well, parallel microfluidic detection device;

FIG. 11B is a chromogenic response of microfluidic device of FIG. 11A that is exposed to RAM antigen; and

FIG. 11C is a chromogenic response of microfluidic device of FIG. 11B (i.e., post RAM testing) that is exposed to GAM antigen (post RAM testing).

### DETAILED DESCRIPTION OF THE INVENTION

Retaining biological activity of the surface-bound molecule, while maintaining and/or enhancing selectivity toward the target analyte molecule, is useful in various applications, such as diagnostic assays. Most conventional antigen detection assays (e.g., standard enzyme-linked immunosorbant assays—ELISAs) rely on monolayer formation or physisorption methods to immobilize antibodies to material surfaces. Unfortunately, the antibody stability, selectivity, and/or uniformity of antibody attachment in these conventional assays are often dependent on material properties, such as surface chemistry and roughness.

Some recent efforts have focused on covalently attaching antibodies to material surfaces through conventional functional groups that are present in the proteins, such as amine and carboxy terminal groups. While these approaches reduce the possibility of antibody desorption, surface-bound antibodies and biomolecules, in general, often lose their activity and/or selectivity due to conformational restrictions, mobility restrictions, and/or mass transfer limitations. Furthermore, antibody activity is often reduced or lost due to protein unfolding or reduction of antigen binding sites due to the coupling process used.

One aspect of the present invention overcomes these limitations by covalently attaching a substrate binding element to a polymer surface. As used herein, the term “substrate binding element” refers to a moiety that binds to any particular substrate with specificity. Exemplary substrate binding elements include ligands and ligand recognition elements that bind to a particular ligand with specificity. As such, the term “substrate binding element” can be either a ligand or its corresponding ligand recognition element, and conversely the term “substrate” refers to its corresponding ligand recognition element or ligand. Ligands include, but are not limited to, antigens, nucleic acid sequences (e.g., RNAs and DNAs including oligomers thereof), peptides (e.g., proteins or a fragment thereof, and protein templates), epitopes, even whole cells, liposomes, and any other molecules that can bind or interact with a particular ligand binding element with at least some level of specificity. Ligand binding elements are those molecules that can bind to or interact with a ligand with specificity such that it can distinguish one ligand from another. Exemplary ligand binding elements include, but are not limited to, enzymes, antibodies, nucleic acids (including RNAs and DNAs including oligomers thereof) that can recognize and bind to a complementary nucleic acid strand, peptides (e.g., proteins or a fragment thereof, and protein templates), liposomes, and any other ligand binding elements that can bind or interact with a particular ligand with at least some level of specificity.

In some embodiments, the substrate binding element is covalently attached to a polymer surface by a covalently attached linker. As used herein, the term “linker” refers to any moiety that is used to attach the substrate binding element to the polymer surface. The linker comprises an optional spacer unit that is attached to a functional group for attaching the spacer unit to a substrate binding element, and a functional group for attaching the spacer unit to a polymer. A spacer unit refers to a polymer or an oligomer that provides a tether such that the substrate binding element remains attached to the polymer. In some embodiments, the spacer unit can be absent in the linker. Suitable spacer units include, but are not limited to, polymers (including oligomers) such as polyalkylene glycols, poly(vinyl alcohols), poly(hydroxy acrylates), poly(hydroxy methacrylates), poly(urethanes), poly(acrylamides), poly(amines), or a combination thereof. Generally, suitable linkers are those that reduce or minimize non-specific substrate binding or attachment to the substrate binding element, linker and/or the polymer surface. Often the linker comprises a hydrophilic portion, such as hydrophilic spacer units.

Exemplary functional groups that can be used to attach the spacer unit (or the linker) to a substrate binding element include, but are not limited to, those functional groups that are known to undergo a covalent bond formation with the functional groups present in the substrate binding element. Such functional groups are well known to one skilled in the art. For example, when the substrate binding element is a protein (e.g., antibodies, enzymes, etc.), functional groups present in the spacer unit (or the linker) to form a covalent bond with the substrate binding element include acrylates, methacrylates, vinyl ether, allyl ether, vinyl silazane, maleate, maleimide, furmarate, allyl isocyanurate, norbornene, as well as other functional groups that can undergo Michael addition or other substitution or conjugate addition reactions.

Similarly, functional groups that can be used to attach the spacer unit (or the linker) to a polymer surface include, but are not limited to, those functional groups that are known to undergo a radical reaction. Such functional groups are well known to one skilled in the art and include, but are not limited to, olefinic moieties, such as acrylates, methacrylates, vinyl ether, allyl ether, vinyl silazane, maleate, maleimide, furmarate, allyl isocyanurate, and norbornene moieties.

In one particular embodiment, the linker comprises a polyalkylene glycol. Polyalkylene glycol of any molecular weight can be used in the present invention. Within this embodiment, typically the linker comprises a polyethylene glycol and/or a polypropylene glycol. Polyalkylene glycols, such as polyethylene glycols, reduce or minimize non-specific protein attachment. Accordingly, polyalkylene glycols are particularly useful linkers when the substrate binding element is an antibody or a protein, an enzyme or other biological material binding element that has specificity to a protein or other biological substrate.

The substrate binding element is covalently attached to the linker through any of the methods known to one skilled in the art, including using the functional groups present in the substrate binding element in a substitution or a coupling reaction to covalently attach the substrate binding element to the linker. Accordingly, in some embodiments, the linker comprises a functional group that is complementary to the functional group present in the substrate binding element such that the substrate binding element can be covalently attached to the linker using any of the conventional methods known to one skilled in the art. It should be appreciated that depending on the number of suitable functional groups present in the linker, more than one substrate binding element can be covalently attached to the linker. Furthermore, the linker can be dendritic-like, thereby allowing a plurality of substrate binding element to be attached at many different branches of the linker.

The linker also comprises another functional group that can be used to covalently attach to the polymer surface. This functional group is preferably of different reactivity such that the reaction between substrate binding element or the polymer surface does not interfere with the reaction between the polymer surface or the substrate binding element, respectively. It should be appreciated that the order of attachment is not critical to the present invention. For example, the linker can be attached to the polymer surface first or it can be attached to the substrate binding element first. Preferably, however, the linker is covalently attached to the substrate binding element first.

It should be appreciated that while the present invention is generally described for assay or detection purposes, the present invention is not limited to such purposes. In fact, methods of the present invention are also useful applications in drug delivery, directed cellular attachment, and bulk network modification, as well as other areas.

The substrate binding element is covalently attached to a polymer surface by a radical reaction. Typically this is achieved by coating the polymer surface with a substrate binding monomer and polymerizing the mixture. The term “substrate binding monomer” refers to a substrate binding element that is covalently attached to a linker, and where the linker comprises a functional group that can react with the polymer surface under, what is believed to be, a radical reaction mechanism. Suitable functional groups on the linker that can be used to attach the substrate binding element to the polymer surface include an olefin. The term “olefin” refers to a carbon-carbon double bond that can undergo polymerization reaction. Exemplary olefinic moieties include, but are not limited to, acrylates, methacrylates, and vinyl moieties. The terms “acrylate” and “acrylate moiety” are used interchangeably herein and refer to a moiety of the formula: H₂C═CH—CO₂—. The terms “methacrylate” and “methacrylate moiety” are used interchangeably herein and refer to a moiety of the formula: H₂C═C(CH₃)—CO₂—. The term “vinyl” and “vinyl moiety” are used interchangeably herein and refer to any non-acrylate or non-methacrylate moiety of the formula H₂C═CH—, accordingly vinyl moiety includes, but are not limited to, allyl moieties, vinyl ether moieties, allyl ether moieties, as well as other olefinic moieties that are not acrylates or methacrylates.

In some embodiments, the linker comprises a vinyl moiety, an acrylate moiety, a methacrylate, or a combination thereof. In one specific embodiment, the linker comprises an acrylate moiety. In another embodiment, the linker comprises a methacrylate moiety. Still in another embodiment, the linker comprises a vinyl moiety.

Polymers can be any polymer that can undergo a radical reaction with the linker. Accordingly, in some embodiments, the substrate binding element is covalently attached to the polymer surface through its covalent attachment to the linker. Suitable polymers of the present invention include polymers disclosed in a commonly assigned PCT Patent application filed on Dec. 16, 2005, entitled “PHOTOLYTIC POLYMER SURFACE MODIFICATION”, which claims the priority benefits of U.S. Provisional Patent Application No. 60/637,111, filed Dec. 16, 2004, and further identified by Attorney Docket Number 66888-327412, which is incorporated herein by reference in its entirety. However, as stated above, any polymer that can undergo a radical reaction with the linker to form a covalently attached substrate binding element can be used. Such polymers include, but are not limited to, polymers comprising a unreacted olefinic or acetylenic moieties, as well as polymers comprising unreacted thiol moieties or halide moieties. Typically, any polymer produced by polymerizing a monomeric mixture comprising a thiol monomer and an olefinic compound can be used. The monomeric mixture can further comprise an iniferter, thereby affording a polymer having iniferter moieties on its surface. In some embodiments, the iniferter is a photoiniferter.

The present invention will be described with regard to the accompanying drawings which assist in illustrating various features of the invention, and are provided for the purpose of illustrating the practice of the present invention and do not constitute limitations on the scope thereof. In this regard, the present invention generally relates to polymers comprising a substrate binding element and a method for using and producing the same. That is, the invention relates to a substrate binding element that is covalently attached to a polymer surface through a linker and methods for using and producing the same.

One particular embodiment of covalently attaching a substrate binding element to a linker is schematically illustrated in FIG. 1. Acrylated, polymerizable antibodies (i.e., “antimers”) are a useful means for covalently attaching antibodies to polymer surfaces for the detection of specific antigens in a given analyte solution. The amount of antibodies that are attached to the linker can be readily determined, for example, by light scattering and a TNBS assay, which monitors the concentration of amine groups before and after attaching to the linker. As can be seen in FIG. 1, antibodies contain various functional groups that can be used to attach to a linker. In FIG. 1, the amino group (—NH₂) represents lysine residue. Thus, reaction between the amino group of a lysine residue with the N-hydroxy succinamide (NHS) moiety of a polyethylene glycol (“PEG”) polymer produces a PEG-attached antibody. In FIG. 1, the PEG polymer also comprises an acrylate moiety that can be used to attach the linker to a polymer surface.

Antimers can be covalently attached within bulk polymer networks and/or to the polymer surfaces, for example, via UV-initiated polymerization reactions. In some embodiments, living radical photopolymerization (LRP) is used to covalently attach the antimer to the polymer surface. The LRP utilizes initiator molecules, called iniferters (in particular photoiniferter), to initiate controlled growth of polymer chains from the polymer surface. Accordingly, LRP can be used to graft (i.e., covalently attach) substrate binding element, for example, whole antibodies, of controlled length, composition, and surface density. Because the antimers are grafted as chains on the polymer surface, the conformation and chain mobility can be tailored by proper selection of the surface grafting linkers and polymerization conditions. Further, the grafting method and composition can be varied to reduce non-specific substrate to substrate binding element interactions and/or to improve solvation of the grafted moiety.

By increasing the mobility and accessibility of binding sites within the polymer surface, substrate to substrate binding element interactions is increased through extension of the substrate binding element into the analyte solution. Enhanced activity and detection limits are achieved in various analyte environments, including plasma and whole blood. Without being bound by any theory, it is believed that such benefits are achieved by overcoming limitations of conventional immobilization techniques with respect to surface mobility and density.

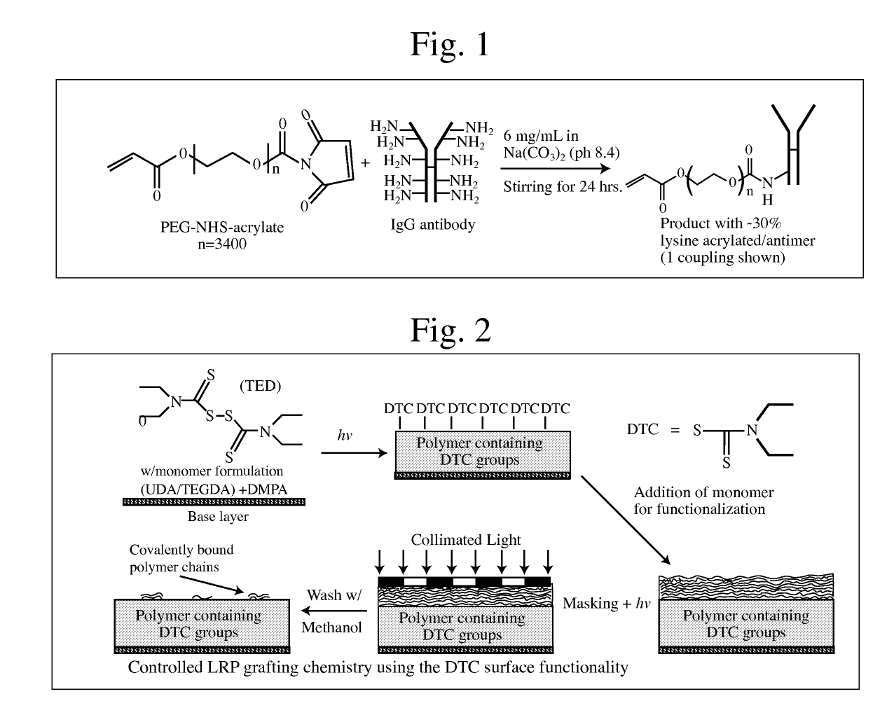

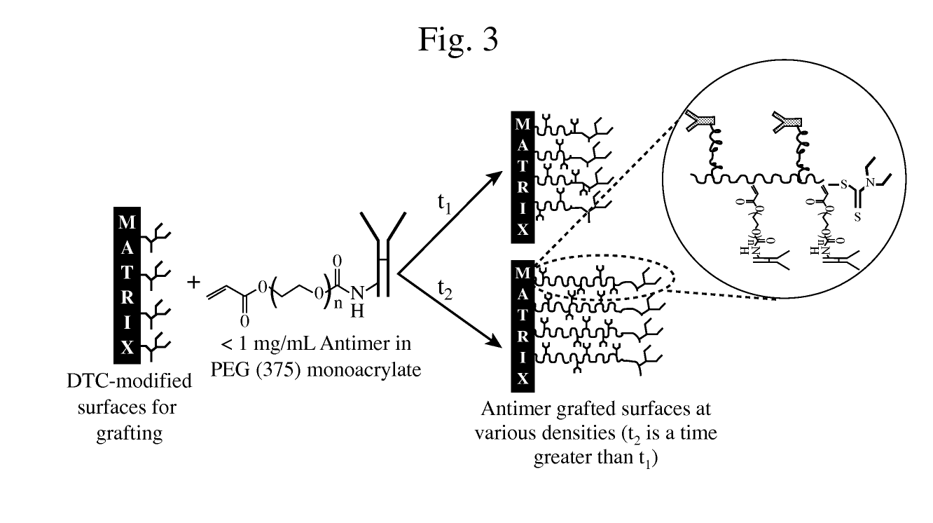

Referring now to FIGS. 2 and 3, the polymer comprising a photoiniferter moiety, e.g., DTC moiety, on its surface is used to graft (i.e., covalently attach) a substrate binding element. As schematically illustrated in FIG. 2, a monomer mixture comprising a thiol monomer, an olefinic monomer, or a combination thereof is cured in the presence of an iniferter (XDT) to form a polymer comprising iniferter moieties on its surface. The polymer is then washed with deionized water and methanol before coating with a substrate binding monomer (i.e., a monomer comprising a substrate binding element that is covalently attached to a linker). Photolithography, which utilizes exposure of the polymer to UV light through a photomask, is used to form patterns of substrate binding elements that are grafted on to reactive surfaces.

Referring again to FIGS. 2 and 3, upon illumination with UV light, the DTC moieties (or other suitable photoiniferter moieties known to one skilled in the art) attached to the polymer surface cleave to generate radicals (typically a reactive carbon radical) on the polymer surface. It should be appreciated that the presence of DTC or other iniferter moiety is not necessary. For example, some thiol groups are known to generate a reactive radical species upon exposure to UV, VIS, γ-ray, x-ray, or other electromagnetic radiation of sufficient energy. Accordingly, by selecting an appropriate thiol monomer, olefinic monomer, or a combination thereof to produce the polymer, one can covalently attach the substrate binding element to the polymer surface without having to have any iniferter present on the polymer surface.

In the presence of a vinyl moiety on the substrate binding monomer, the reactive radicals generated react with the vinyl moiety to form covalently attached substrate binding element that is tethered to the polymer surface by the linker. In the case of monoacrylates, the graft length can be controlled by the exposure time, thereby allowing the degree of surface graft control. This approach enables covalent binding of the antibodies with independent control over their density and clustering, which can improve substrate (e.g., antigen) detection sensitivity and response time. As can be seen, whole antibodies can be attached to polymer surfaces. Using the whole antibody allows substantially all of its activity and selectivity to be maintained. Moreover, use of the whole antibody allows selectivity in a variety of biologically samples (e.g., relevant analyte environments).

Another aspect of the present invention provides microfluidic devices and methods for producing the same. Microfluidic devices and methods for producing them are well known to one skilled in the art. See, for example, U. S. Patent Application Publication No. 20050129581, published Jun. 16, 2005, and references cited therein, all of which are incorporated herein by reference in their entirety. Microfluidic devices can be used to perform various chemical and biochemical analyses. There are significant benefits to use of microfluidic devices because of their miniaturization in size. Such benefits include a substantial reduction in time, cost and the space requirements for the devices utilized to conduct the analysis. Additionally, microfluidic devices have the potential to be adapted for use with automated systems, thereby providing the additional benefits of further cost reductions and decreased operator errors because of the reduction in human involvement. Microfluidic devices of the present invention comprise at least a portion of the channel that comprises a covalently bound substrate binding element. As used herein, the term “surface” refers to any area of the polymer that one skilled in the art can consider to be in contact with ambient atmosphere. Accordingly, for microfluidic devices or any other polymers with channels or porous polymers, the term “surface” includes surfaces that surround and define the channels of microfluidic devices as well as interstitial surfaces which are the surfaces that surround and define the pores of polymers.

Typically, microfluidic devices are formed by producing one layer at a time and attaching one layer to another. For example, a first layer of polymer is produced with a desired channel pattern(s) and/or substrate binding element(s) attached to a desired portion of the channel(s). A second polymer layer is produced and is bonded to the first layer. Such bonding of the two layers can be achieved by using any of the methods known to one skilled in the art, for example, by reacting the first layer with the second layer to form covalent bond, or using an adhesive to bind the first layer to the second layer. By attaching more layers, microfluidic devices having a complex channel pattern can be produced. Microfluidic devices of the present invention provide highly efficient, rapid (e.g., 5-12 minutes or less), parallel screening of analyte. Such a rapid analysis is a significant improvement over conventional immunoassays, which takes hours or even days.

Methods of the present invention provide photografting of one or more, dense substrate binding elements, with improved sensitivity and specificity relative to the conventional methods. The term “photograft” refers to covalently attaching a monomer to a polymer using an electromagnetic radiation, such as IR, Vis, UV, x-ray, or γ-ray. Some methods of the present invention provide simple integration of any substrate binding element, e.g., antibody, onto polymer surfaces. Methods of the present invention allow one to control the area and density of covalently attached substrate binding element, for example, through the use of a photolithography or a LRP process. Without being bound by any theory, it is believed that increased sensitivity of devices of the present invention (polymers, microfluidic devices, as well as any other devices fabricated using methods of the present invention) is believed to be associated with grafted substrate binding elements (e.g., antibodies) that are covalently polymerized to form mobile (i.e., non-rigid) polymer chains that provides increased substrate binding element accessibility.

The present invention is applicable in covalently attaching growth factors, other proteins, cell sensing moieties, as well as any other suitable substrate binding elements, on or within polymeric matrices. As stated herein, methods of the present invention provide many advantages over conventional methods, such as by controlled polymerizations, substrate binding element density and clustering can be tailored for increased sensitivity. Devices and methods of the present invention also allow for either qualitative or quantitative analysis without the requirement of any expensive or sophisticated equipment, such as well-plate readers or a UV-Vis spectrophotometer. Some devices and methods of the present invention allow quantification of assay result simply by using an average digital scanner and readily available, free-of-charge imaging software. Additionally, the ability to visualize results easily, combined with the portable size of polymers and microfluidic devices of the present invention and the stability of the covalently attached antimers (i.e., substrate binding element with a covalently linked linker), allow assays to be performed in the field (i.e., directly at the site) rather than having to send the sample to any particular location for analysis. In addition, devices and methods of the present invention provide an efficient means for on-the-spot screening and detection of various biological agents, including molecules such as glucagon, that have short half-lives in plasma and whole blood.

It has been found by the present inventors that when a substrate binding elements, such as antibodies, are covalently attached according to the present invention, the antibodies retain substantial amount of their biological activity and specificity for rapid antigen detection. Typically, at least 50% of the antibody activity is retained, i.e., relative to its unbound form. Preferably, at least 75%, more preferably at least 80% and still more preferably at least 90% of the antibody activity is retained.

As expected, the sensitivity of assay depends on the density of the substrate binding element that is covalently attached to the polymer surface. Generally, methods of the present invention provide polymers that can have nanomolar or even picomolar sensitivity. Furthermore, methods and devices of the present invention are useful for the detection of short half-life substrates, e.g., antigens, or those that need to be detected in less than 20 minutes. It is believed that the combined sensitivity and short assay time are the results of the ability to surface immobilize the antibodies on grafted chains (i.e., linkers) of controlled length and composition of the linkers. In addition, present invention eliminates time-consuming blocking steps and non-specific protein interactions associated with standard assay techniques, such as ELISAs. For example, using a linker comprising PEG, such as PEG-375, reduces or inhibits protein adhesion; therefore, there is no need to block non-specific antigen adhesion sites, which eliminates a lengthy step involved in most antibody-antigen binding assays.

Additional objects, advantages, and novel features of this invention will become apparent to those skilled in the art upon examination of the following examples thereof, which are not intended to be limiting.

### EXAMPLES

### Materials

Antibodies including donkey anti-goat (DAG), rabbit anti-mouse (RAM), goat anti-rabbit (GAR), goat anti-rabbit HRP (GAR-HRP) and goat anti-mouse HRP (GAM-HRP) were purchased from ICN Biochemicals, Inc. (Irvine, Calif.). Monoclonal anti-glucagon (GLGN) was purchased from Sigma-Aldrich (St. Louis, Mo.). Glucagon antigen was purchased from Calbiochem (Jolla, Calif.). The antibody peroxidase labeling kit was purchased from Roche (Indianapolis, Ind.). The 3,3′-5,5′-tetramethylbenzidine (TMB) staining kit was purchased from Corgenix Corp. The Vector VIP substrate was purchased from Vector Labs (Burlingame, Calif.). Trinitrobenzene sulfonic acid was purchased from Pierce (Rockford, Ill.). Sterile bovine plasma and calf blood in Alsevers was purchased from Rockland Immunochemicals (Gilbertsville, Pa.).

Urethane diacrylate (UDA) Ebecryl 4827 was obtained from UCB Chemicals Corp (Smyrna, Ga.). Triethyleneglycol diacrylate monomer (TEGDA) was purchased from Sartomer (West Chester, Pa.). Poly(ethylene glycol (375)) monoacrylate (“PEG-375 acrylate”) and tetraethylthiuram disulfide (TED) were purchased from Sigma-Aldrich (St. Louis, Mo.). Poly(ethylene glycol)-acrylate-N-hydroxysuccinimide MW 3400 (ACRL-PEG-NHS) was purchased from Nektar Therapeutics (Birmingham, Ala.). The initiator 2,2-dimethoxy-2-phenylacetophenone (DMPA) was purchased from Ciba Specialty Chemicals (Tarrytown, N.Y.).

### Example 1

Each of the whole antibodies was acrylated using NHS:NH₂ conjugation according to the following procedure. The antibody was dissolved (6 mg/mL) in 50 mM sodium bicarbonate solution, pH 8.4, and reacting the antibody amine groups with ACRL-PEG-NHS, MW 3400 in a variety of molar ratios ranging from 0.1 to 2.0 (NHS:NH₂). The reaction was allowed to proceed for 3 hours at room temperature with shaking. Excess ACRL-PEG-NHS and other reaction byproducts were removed via dialysis against deionized water for 24 hours (MWCO 10,000), followed by lyophilization, resulting in a solid product.

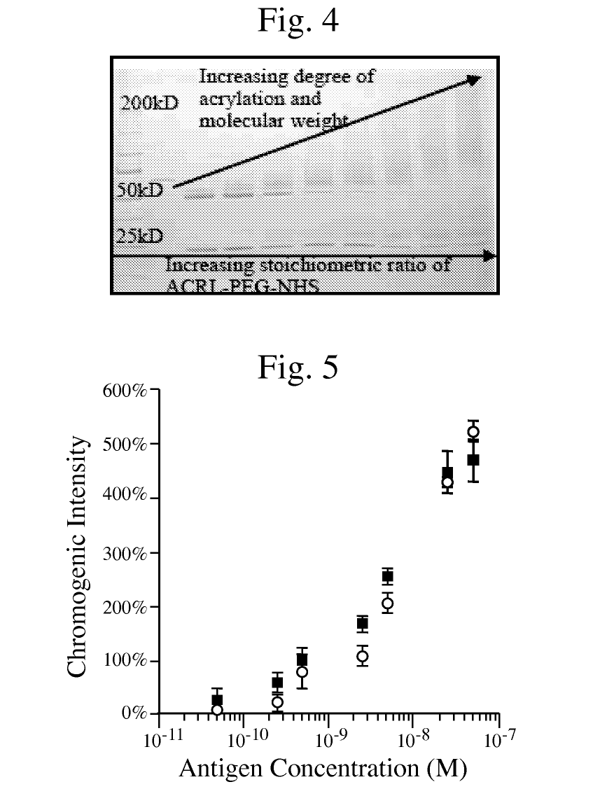

Antibody acrylation was verified via SDS-Page and a trinitrobenzene sulfonic acid (TNBS) assay. See, for example, Hermanson, Bioconjugate Techniques, Elsevier Science and Technology Books, San Diego Calif., 1996, p. 112; and Wild, The Immunoassay Handbook, (2001) Nature Pub. Group, 2nd Ed. The degree of acrylation was determined using a standard TNBS assay protocol, monitoring the concentration of amine groups before and after conjugation chemistry was performed. Also, antibody digestion of disulfide bridges was performed to yield heavy (˜50 kD)) and light (˜25 kD) fragments. Then, SDS-Page was used to determine approximately which regions of the antibody were being conjugated and to further estimate the molecular weight of the fragments after reacting with a range of stoichiometries of 0.1-2.0 NHS:NH₂ to perform a range of conjugations. As shown in FIG. 4, the increase in acrylation was associated with an increased ACRL-PEG-NHS in the reaction stoichiometry. Although the SDS-Page results indicated conjugation on both heavy and light chains, the results did suggest a much higher degree of acrylation on the heavy chain fragments of the antibody (bands starting at 50 kD). This result implies that less modification was occurring in the light chain region of the antibody, leading to less interference with the antibody hypervariable region, associated with retention of antibody specificity.

Exemplary antibodies that were acrylated using this procedure include, but are not limited to, affinity purified goat anti-rabbit IgG (GAR), horseradish peroxidase conjugated goat anti-rabbit IgG (GAR-HRP), affinity purified donkey anti-goat IgG (DAG), goat anti-mouse IgG-HRP (GAM-HRP), and anti-glucagon (GLGN) were acrylated. It was determined that the degree of acrylation for each of these antibodies was about 30%. The average particle diameter was observed using light scattering technique and was determined to be about 84 nm. Without being bound by any theory, it is believed that the particle size is due to protein aggregation. The average particle diameter of the antimer particles at the same concentration following photopolymerization for 30 minutes was about 378 nm. It is believed that increase in average particle size upon UV-photopolymerization is due to chain polymerization of acrylated antimers.

### Example 2

Activity of modified (i.e., acrylated) antibodies of Example 1 was determined by using a standard ELISA protocol for the detection of a variety of antigen concentrations in PBS (5.0×10⁻¹¹ M-5.0×10⁻⁸ M). RAM antigen was detected and compared to detection using both unmodified primary GAR and acrylated GAR antibody. The ELISAs were carried out on Immulon High-Binding 96-well plates. Each well was coated at 4° C. overnight with 100 μL of primary detection antibody (affinity purified, acrylated and unmodified GAR) dissolved in 0.1 M sodium bicarbonate solution (pH 9.4) at a concentration of 5 μg/mL. After aspirating off coating buffer and unattached primary antibody, wells were washed with 200 μL of wash buffer (PBS+0.1 v/v Tween-20). Each well was blocked for 2 hours at room temperature, using 200 μL of blocking buffer (PBS+5 mg/mL fraction V BSA). After blocking, each well was washed 4 times with 200 μL of wash buffer. Then, 100 μL of RAM antigen in PBS (5.0×10⁻¹¹ M-5.0×10⁻⁸ M) was added to each well and incubated for 1 hour, followed by washing. After washing, 100 μL of secondary GAR-HRP (5 μg/mL) was added to each well and incubated for 1 hour. Following appropriate washing, the wells were then tested for antibody activity using a plate reader (Victor², Perkin Elmer) at 450 nm to detect activity between surface grafted antibody-containing chains and the complementary, enzymatic substrates. Activity was quantitatively analyzed through the use of TMB, a peroxidase substrate. Reaction of TMB with HRP resulted in a soluble blue product after 30 minutes, thereby creating a visually detectable chromogenic response in solution. At this point, the reaction between the TMB and the HRP-labeled antibody graft was terminated through the addition of an equal volume of 0.36 N sulfuric acid, turning the TMB solution yellow for quantitative analysis by the spectrophotometer. ELISAs for other antibodies were carried out using this procedure.

As shown in FIG. 5, only a slight decrease in the acrylated antibody (∘) activity was observed compared to ELISAs carried out with non-acrylated GAR (□) using the same ELISA protocol. Further studies using standard ELISAs were completed to determine if the specificity of acrylated GAR antibodies was maintained for detection of non-specific mouse antigen. Specificity results showed no statistically significant decrease in specificity when comparing modified and unmodified GAR antibodies.

### Example 3

Polymers were prepared from monomer formulations consisting of 48.75 wt % aromatic UDA and 48.75 wt % TEGDA mixed with 1 wt % TED and 1.5 wt % DMPA initiator. The formulations were sonicated for 45 minutes and purged with argon gas for 2 minutes prior to photopolymerization. The polymer (about 300 μm of thickness per layer) was then photopolymerized by exposure to a 45 mW/cm² intensity collimated, broad-range UV light (Hg arc-lamp centered at 365 nm) for 500 seconds. After photopolymerization in the presence of TED, the resulting UDA/TEGDA, crosslinked polymer had photolabile dithiocarbamate (DTC) groups that were further used to reinitiate the formation of surface-attached (i.e., covalently bound), photografted chains through living radical photopolymerization (LRP) chemistry. These exposure conditions gave a polymeric network with over 90% double bond conversion, as observed when monitoring the acrylate double bond absorbance peak, using near-IR analysis. See, for example, Hutchison et al., Lab Chip, 2004, 4, 658-662. After polymerization, the polymers were washed with copious amounts of methanol to remove any unreacted species prior to surface modification.

### Example 4

Acrylated antibody (“antimer”) was covalently photografted to a polymer surface using the LRP surface chemistry. A solution containing 0.1 mg of acrylated antibody (includes mass of any protein impurities) was mixed with 1 mL of PEG-375 monoacrylate solution for 10 minutes and purged with argon for 2 minutes before grafting. A patterned region of grafted antibody/PEG-375 acrylate was formed upon exposure to 45 mW/cm² intensity UV light for 900 seconds using photolithographic techniques disclosed by Hutchison et al., in Lab Chip, 2004, 4, 658-662, which is incorporated herein by reference in its entirety. The resultant pattern was washed in 50/50 ethanol/water and then deionized water for 24 to 48 hours.

### Example 5

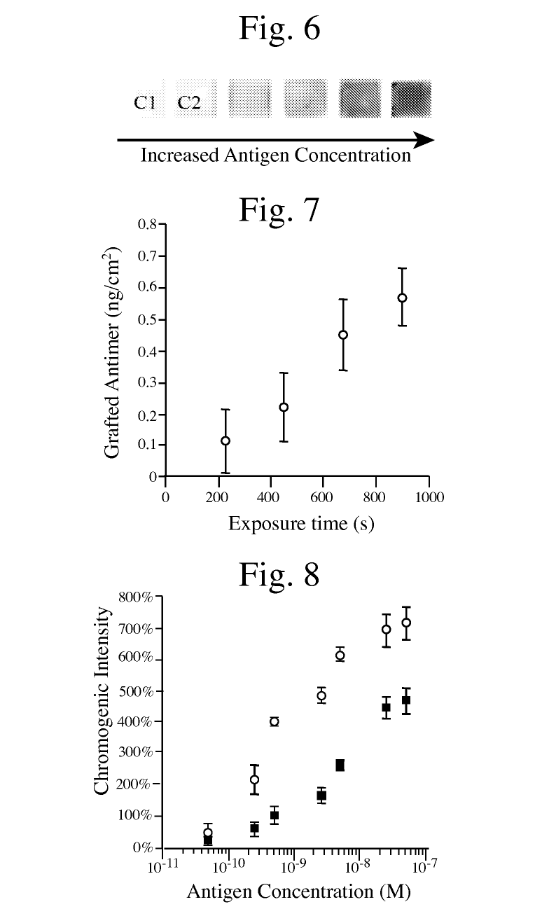

GAR antibody was dissolved in PEG-375 monoacrylate at various concentrations (pM to nM). Using LRP-based grafting process, acrylated antibody was covalently attached in 15 minutes, via UV photografting, to a polymeric surface containing the DTC moiety. Photografted GAR was exposed to a various concentrations of rabbit antigen (5.0×10⁻¹¹ M-5.0×10⁻⁸ M). The polymer was assayed with a Vector VIP to illustrate visually that individual surface-bound detection squares (5 mm×5 mm) were formed arid maintained their binding capabilities. FIG. 6 shows photografted, square patterns of antibody-PEG acrylate that increase in chromagenic intensity with increasing antigen concentration compared to control surfaces containing only PEG grafts (C1) and PEG grafts synthesized in the presence of non-acrylated antibody (C2).

Other experiments also confirmed that the antibody density in photografted chains was controlled by the concentration of acrylated antibodies in the grafting solution, as well as the photopolymerization time. Significant increases in GAM-HRP graft density were achieved by increasing the UV exposure time, up to 15 min., which increased the surface concentration of grafted antibody in a nearly linear fashion from 0.10-0.57 ng antibody/cm². Also, by varying the concentration of acrylated antibody in the grafting solution, the surface graft composition can be controlled, as determined by chromogenic development of HRP using TMB. A consistent increase in antibody surface composition was observed with increasing GAM-HRP concentration in PEG solution (0-0.10 mg antibody/mL soln.) as compared to control PEG grafts in the presence of non-acrylated antibody (results not shown).

### Example 6

Other antibodies were acrylated and covalently attached (i.e., grafted) to a polymer surface using procedures described herein. For example, acrylated GAR was patterned to form individual detection squares (5 mm×5 mm) on the polymer. After the specified reaction time, detection squares were rinsed 4 times with 100 μL of phosphate buffer solution (PBS) to remove any unbound antibodies. As can be seen in FIG. 7, the density of antimer attachment to the polymer surface increased with exposure times. After exposing grafted samples to RAM antigen at various concentrations in PBS (5.0×10⁻¹¹ M-5.0×10⁻⁸ M) for 5 minutes at 37° C., the polymer samples were washed and exposed to secondary GAR-HRP (5 μg/mL) for 2 minutes at 37° C. After washing 4 times with PBS, bound antigen was detected calorimetrically by GAR-HRP reaction with Vector VIP staining kit for 5 minutes. This reaction resulted in a surface-bound chromogenic response, where the amount of bound, HRP-labeled analyte was proportional to the intensity of the resulting purple color. After rinsing the sample with PBS, quantitative grayscale analysis was performed using a digital scanner (Hewlett Packard ScanJet 4100C) and NIH Scion Image analysis software. Control polymer samples consisted of a PEG acrylate grafted polymer (with non-acrylated antibody washed away) assayed under the same conditions and a polymer sample that was grafted using equal concentrations of antibody in the grafting solution but was not exposed to antigen during the assay procedure.

### Example 7

The reaction time required to sufficiently observe binding between antibody (GAR) and a labeled antigen (RAM-HRP) was determined by exposing (i.e., contacting) surface bound antibody to a 5 μg/ml solution of RAM-HRP in phosphate buffer solution (equal to 100 ng antigen/square) for 2, 5, or 10 minutes. Bound antigen was detected colorimetrically by RAM-HRP reaction with a Vector VIP staining kit for 5 minutes. This reaction resulted in a surface-bound chromogenic response, where the amount of bound, HRP-labeled antigen was proportional to the intensity of the resulting purple color. The surface-bound chromogen (Vector VIP) was used to prevent chromogen diffusion when integrating this method onto a microfluidic device. Compared to a negative control of RAM-HRP reacted with a PEG-only graft, a 2 minute reaction time resulted in a 25-fold increase in chromogenic response intensity, while at 5 minutes, the response was 50 times that of the control. The reaction achieved a maximum response after 5 minutes, as further reaction time did not significantly increase the response intensity.

### Example 8

GAR-grafted antibody squares were also reacted with various concentrations of antigenic analyte to investigate the detection limits. The detection limit was determined as an intensity that was statistically significant compared to that of a PEG grafted square tested at the same conditions. GAR-grafted samples were prepared and reacted with a range of dilutions of RAM in PBS (5.0×10⁻¹¹ M-5.0×10⁻⁸ M). After exposure to the secondary antibody, this procedure effectively formed an analyte “sandwich”, as antigen is bound to both the surface-tethered antibody and the HRP-labeled GAR. As shown in FIG. 8, which shows chromagenic intensity as a function of antigen concentration for detecting RAM using a standard ELISA technique (□) and using grafted antibody (O), the detection limit of this technique was less than 0.1 nM of antigen in analyte solution and was comparable with ELISA results, even though detection was performed in about 15 minutes. Decreasing antigen concentration led to a decrease in colorimetric response as expected p<0.0001). Due to less background noise on control samples, the chromogenic response for the sandwich assay performed with the grafted antibody method was typically much higher than that for the corresponding antigen concentration when using standard ELISA techniques.

### Example 9

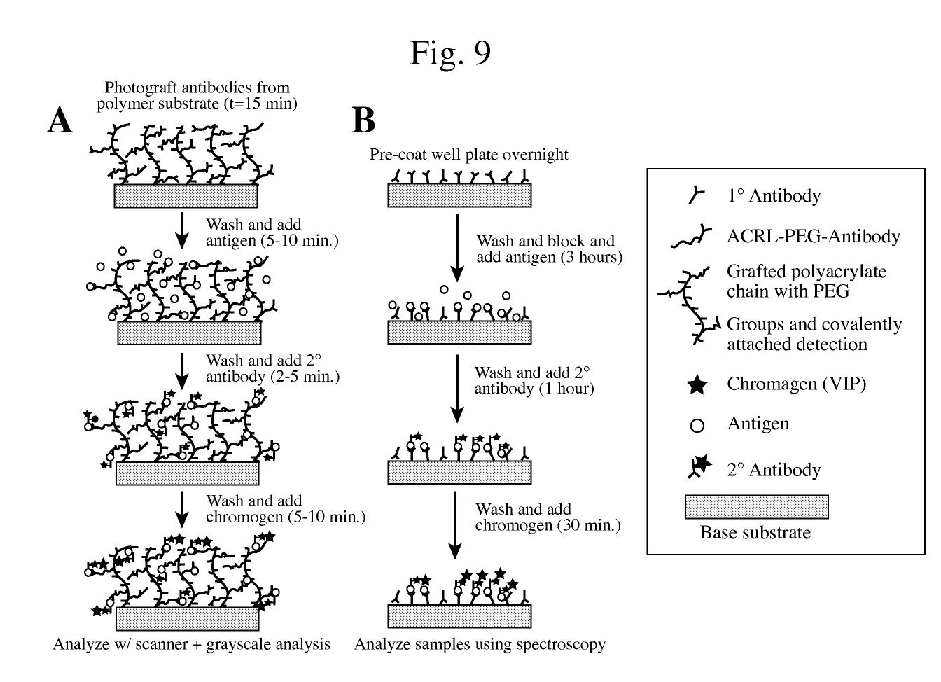

GAR-antibody grafted polymer samples were prepared using the procedures described herein. After the appropriate washing was completed, a range of RAM dilutions (5.0×10⁻¹¹ M-5.0×10⁻⁸ M) was prepared in PBS, and 100 μL was placed on each antibody-grafted polymer sample square and incubated at 37° C. for 5 minutes. After 4 washes using PBS, 100 μL of GAR-HRP at a concentration of 5 μg/ml was added to each sample and allowed to react with the RAM for 2 minutes. After further washing with PBS, Vector-VIP enzymatic chromogen was added and allowed to react for 5 minutes at 37° C. Polymer samples were rinsed and calorimetrically analyzed using a scanner and NIH image analysis software. Control polymer samples consisted of a PEG acrylate grafted polymer (with non-acrylated antibody washed out) assayed under the same conditions and a polymer sample that was grafted using equal concentrations of antibody in the grafting solution but was not exposed to antigen during the assay procedure. These results were then compared to ELISA results that were determined using the standard ELISA protocol. The comparative procedures of these two methods are schematically illustrated in FIGS. 9A and 9B, which show methods of the present invention and standard ELISA assay, respectively.

### Example 10

Glucagons (GLGN) is a 29 amino acid peptide sequence that opposes the effects of insulin in gluconegenesis and glycogenolysis. It has a relatively short half-life (<15 minutes) in whole blood. Therefore detection of GLGN requires a short assaying time.

Anti-GLGN grafted polymer samples were prepared using the procedures described herein using a grafting solution concentration of 1.0 mg/mL anti-GLGN acrylated antibody in PEG. A range of GLGN dilutions was prepared in PBS, 20% whole blood in PBS, and 20% plasma in PBS. These solutions were tested quickly (12-15 minutes) on anti-GLGN grafted polymer samples immediately after washing.

GLGN-HRP was synthesized using a peroxidase labeling kit, purified using ultrafiltration, and then added to the GLGN dilutions at a concentration of 5 μg/mL and allowed to react with the GLGN antigen for 2 minutes. After 5 minutes of reaction time at 37° C., polymer samples were rinsed and calorimetrically analyzed using Vector VIP chromogen exposure for 5 minutes. Grayscale analysis was then used to quantify results. Control polymer samples consisted of GLGN-grafted polymer squares exposed to samples containing GLGN-HRP but no antigen and a PEG-acrylate only polymer sample. GLGN ELISA data was gathered using the standard ELISA protocol.

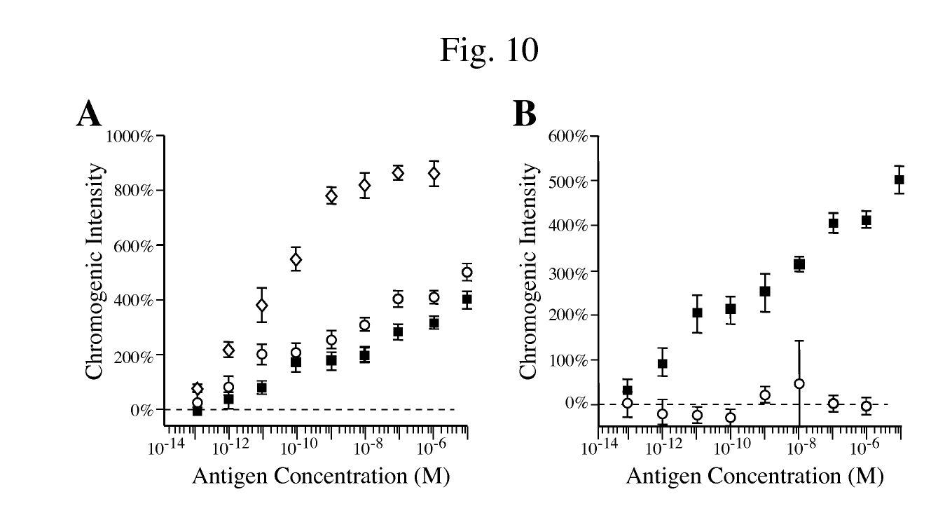

FIG. 10A shows chromogenic intensity of Vector VIP (after 5 minutes) as a function of GLGN antigen concentration in PBS (⋄), 20% plasma (∘), and 20% whole blood (□) analyte. Values in FIG. 10A are reported as a percentage intensity increase relative to control sample intensities in the absence of antigen (GLGN). The detection limits are shown as the point at which the sample intensity was not statistically different from that of a negative control (---) (1.0 pM for blood and plasma-containing samples and ˜0.5 pM for the assay carried out in PBS). The detection limit of GLGN in PBS using the grafted antibody immunoassay was determined to be around 1.0×10⁻¹³ M GLGN antigen in PBS analyte solution. In whole blood and plasma, detection intensities were decreased by 66% and 53%, respectively; however, as can be seen in FIG. 10A, intensities remained significant and the detection limits remained comparable (pM). As a comparison, an attempt to detect GLGN in plasma using standard ELISA techniques was made. The comparative assays results are shown in FIG. 10B, where (□) represents chromogenic intensities for grafted antibody immunoassay and (O) represents chromogenic intensities for standard ELISA. As can be seen, chromogenic intensities for standard ELISA assays were insignificant throughout this range of GLGN dilutions.

### Example 11

Microassays with antibody-grafted detection wells were constructed using polymer materials described herein. First, a base layer was polymerized as described in Example 3 on a polycarbonate base. Then, a high-resolution photomask was placed in contact with argon purged, monomeric matrix solution. The thickness was adjusted to 300 μm prior to collimated flood exposure, leading to a spatially controlled polymer layer atop the previous polymer layer. After the UV exposure, unreacted monomer was removed via a methanol wash, and the polymerized trenches (channels) were filled with wax to prepare a level surface for further polymerization of sequential layers within the polymeric device. See Hutchison et al., Lab Chip, 2004, 4, 658-662. Each 2 mm well was photografted for 900 seconds with either control PEG-375 monoacrylate monomer or specific antibody-PEG detection monomer for detection purposes. The microassay was removed from a polycarbonate base and void regions were cleared, resulting in a filly polymeric microassay sample, modified with surface chemistries for detection purposes. Photografted antibody was used to modify individual wells for selective detection of RAM-HRP and GAM-HRP antigen.

### Example 12

GAR and DAG antibodies were acrylated and purified via the coupling procedure described for GAM-HRP. For this particular set of experiments, 2 mm diameter wells on the second layer of the microassay were modified with 1.0 mg of GAR acrylate (includes mass of protein impurities) dissolved in 1 mL PEG-375 for grafting, after exposure to UV light for 900 seconds. Similarly, a well was also modified with DAG antibody.

To verify device activity and specificity, a device with 2 mm diameter wells grafted with GAR acrylate was tested. The device was placed into a solution of 5.0 μg/mL HRP conjugated RAM antibody solution for binding with the surface attached antibody. The device in solution was also incubated at 37° C. for 10 minutes. After this procedure, the wells were analyzed for surface activity, using methods described herein involving the reaction between Vector VIP and HRP. Similarly, detection of GAM-HRP was achieved sequentially. For smaller device wells the assay time was 30 min. to amplify the detection intensity for better visualization.

### Example 13

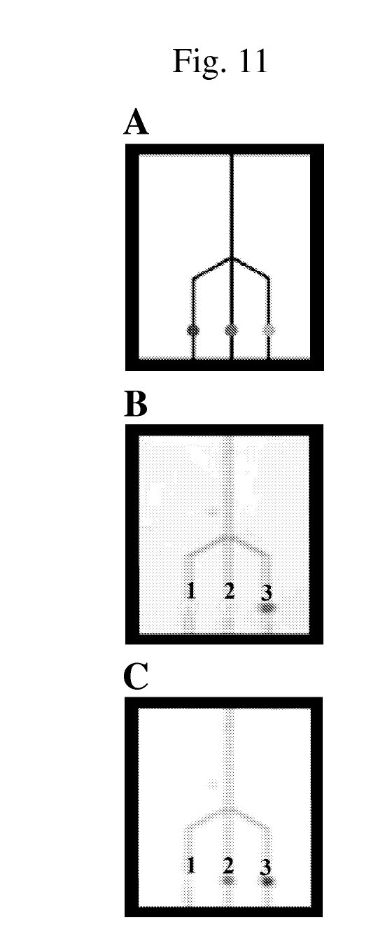

Using the LRP-based antibody grafting combined with a photolithographic technique (see Hutchison et al., Lab Chip, 2004, 4, 658-662), a fully polymeric microfluidic device with antibody-grafted detection channels was constructed. For the design shown in FIG. 11A, 2 mm diameter channels incorporated on a microfluidic device were modified as follows: one channel (left, channel 1) with PEG-375 monoacrylate only, one channel (middle, channel 2) with a mixture of DAG acrylate dissolved in PEG-375 monoacrylate for grafting, and one channel (right, channel 3) with GAR acrylate dissolved in PEG-375 monoacrylate for grafting. After proper washing, the antibody-modified channels of the microfluidic device were exposed to various antigens to demonstrate both the specificity of the covalently immobilized antibodies, and the ability to use a microfluidic device to perform parallel detection of multiple analytes using this microfluidic device grafted with different antibodies on different channels.

A 0.1 mg/mL solution of GAM-HRP in PBS was exposed to all channels for 10 minutes and reacted with Vector VIP. This reaction yielded a positive response in channel 3 as shown in FIG. 11B, corresponding to the anti-goat antibody bound to channel 3. Cross-reactivity on non-specific channels was negligible and did not lead to a false positive result. Following this reaction, the channels were exposed to a 0.1 mg/mL solution of RAM-HRP under the same reaction conditions. Channel 2, containing an anti-rabbit antibody, developed a positive response due to specific detection of the rabbit-based antibody, as shown in FIG. 11C. This result demonstrates the ability to detect multiple antigens in parallel channels on the same microfluidic device. Further, the control channel (grafted with PEG-375 monoacrylated only) that was not specific to the exposed antigens provided an insignificant chromogenic response when tested, illustrating the ability to prevent significant non-specific protein adhesion to microfluidic devices made by grafting with the PEG-375 acrylate.

### Example 14

Data sets were compared using single factor ANOVA tests. P− values <0.05 were considered significant. All chromogenic intensity data was normalized to control samples where ELISA protocol or photografted antibody immunoassay protocol was used in the absence of antigen to eliminate concern related to false positives and non-specific antibody activity.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A method for covalently attaching a substrate binding element to a polymer surface, wherein the polymer surface comprises a first polymerizable functional group, said method comprising:
coating the polymer surface with a substrate binding monomer to produce a polymerizable mixture, wherein the substrate binding monomer comprises a substrate binding element that is covalently attached to a linker comprising a second polymerizable functional group; and
polymerizing the polymerizable mixture to produce a polymer comprising covalently attached substrate binding monomer.

2. A polymer produced from a monomer mixture comprising a thiol monomer and an olefinic monomer, said polymer comprising a surface bound substrate binding element, wherein said substrate binding element is covalently attached to said polymer surface by a covalently attached linker.

3. A method for producing a polymer comprising a covalently attached substrate binding element, said method comprising polymerizing a mixture of a first monomer and a substrate binding monomer by a radical polymerization reaction to produce a polymer comprising a covalently attached substrate binding element, wherein the first monomer comprises a first polymerizable functional group and the substrate binding monomer comprises a substrate binding element that is covalently attached to a linker comprising a second polymerizable functional group such that the radical polymerization reaction results in formation of a covalent bond between at least a portion of the first polymerizable functional group and at least a portion of the second polymerizable functional group by a radical polymerization process.

4. A microfluidic device comprising:
a polymer derived from a monomeric mixture comprising a thiol monomer and an olefinic monomer; and
a plurality of channels, wherein a surface of at least a portion of one of the channels comprises a surface bound substrate binding element, wherein said substrate binding element is covalently attached to said surface by a covalently attached linker.

5. A device for use in a biological assay comprising a polymer produced from a monomer mixture comprising a thiol monomer and an olefinic monomer; and wherein at least a portion of the polymer surface comprises a covalently attached substrate binding element, wherein said substrate binding element is covalently attached to the polymer surface by a linker.

6. The polymer of claim 2, wherein said linker comprises polyethylene glycol, poly(vinyl alcohol), poly(hydroxy methacrylate), poly(hydroxy acrylate), poly(urethane), poly(acrylamide), poly(amines), or a combination thereof.

7. The polymer of claim 6, wherein said linker comprises polyethylene glycol.

8. The polymer of claim 2, wherein said substrate binding element is an antibody.

9. The polymer of claim 2, wherein said substrate binding element is an antigen.

10. The polymer of claim 2, wherein said substrate binding element is a cell.

11. The polymer of claim 2, wherein said polymer comprises a plurality of surface bound substrate binding elements, wherein each of said substrate binding element has different binding affinity for different substrates.

12. The polymer of claim 2, wherein each of said surface bound substrate element is covalently linked to a portion of the polymer.

13. The polymer of claim 2, wherein the polymer surface is photoreactive.

14. The polymer of claim 2, wherein said polymer comprises a photoiniferter moiety.

15. The polymer of claim 2, wherein said substrate binding element is photografted or photopolymerized to said polymer surface.

16. The method of claim 1, wherein the first polymerizable functional group comprises an iniferter moiety.

17. The method of claim 1, wherein the polymer is produced from a monomeric mixture comprising a thiol monomer, an olefinic monomer, or a combination thereof.

18. The method of claim 3, wherein the first monomer comprises a thiol monomer, an olefinic monomer, or a mixture thereof.

19. A polymer comprising a substrate binding element covalently attached within the polymer bulk matrix.

20. The method of claim 3, wherein the first polymerizable functional group comprises an iniferter moiety.
